(19) United States
(12) Patent Application Publication
(10) Pub. No.: US 2015/0342760 A1
(43) Pub. Date: Dec. 3, 2015

(54) ANTI-MIGRATION STENT

(71) Applicant: Boston Scientific Scimed Inc., Maple Grove, MN (US)

(72) Inventors: Laura Elizabeth Christakis, Worcester, MA (US); Michelle Fater, Worcester, MA (US)

(73) Assignee: Boston Scientific Scimed Inc., Maple Grove, MN (US)

(21) Appl. No.: 14/722,534

(22) Filed: May 27, 2015

Related U.S. Application Data
(60) Provisional application No. 62/006,332, filed on Jun. 2, 2014.

(51) Int. Cl. A61F 2/82; A61F 2/95

(52) U.S. Cl. CPC A61F 2/82; A61F 2/95; A61F 2210/0076; A61F 2210/0004; Y10S 623/00; Y10S 977/905

(57) ABSTRACT

Embodiments of the disclosure relate to devices, systems and methods of use and manufacture. One such device is a stent. The stent includes a stent body and a coating over at least a portion of the stent body. The coating includes a shape memory polymer having an outer surface. The outer surface defines a first configuration and a second configuration. In the first configuration the outer surface is smooth. In the second configuration, the outer surface includes a micro pattern.

### CROSS-REFERENCE TO RELATED APPLICATIONS

This Application claims the benefit of and priority to U.S. Provisional Application No. 62/006,332, filed Jun. 2, 2014, the entire contents of which are herein incorporated by reference.

### TECHNICAL FIELD

The present disclosure pertains to an intraluminal prosthesis having an anti-migration feature, and methods for using and manufacturing the same. More particularly, the present disclosure pertains to stent having a coating as an anti-migration feature.

### BACKGROUND

Stents are typically small mesh like structures that can be used to treat blocked areas, such as arteries, within a patient's body. Some stents may be coated with medicine, which is released over a period of time. Typically, the stents may be categorized as permanent, removable, or bioresorbable. Permanent stents are retained in place and incorporated into the lumen wall of the body. Removable stents may be removed from the body lumen when the stent is no longer required for treatment. Bioresorbable stents may be composed of, or include, biodegradable material or bioresorbable material, which may be broken down by the body and absorbed or passed from the body when it is no longer required. The removable stents may be preferable as compared to permanent stents in treating many bodily vessels, such as many esophageal stenosis procedures that require stent removal at specified dates/times.

### SUMMARY

A stent with enhanced anti-migration features to resist, impede or prevent migration, and which can be easily removed after a certain period, pursuant to treatment requirements, is herein disclosed. At least some embodiments are therefore directed to stents containing a bioadhesive thermoplastic or heat activated shape memory polymer coating that enhances anti-migration.

In at least one embodiment, a stent includes a stent body and a coating over at least a portion of the stent body. The coating includes a shape memory polymer defining an outer surface. The outer surface of the shape memory polymer defines a first configuration and a second configuration. In the first configuration, the outer surface is smooth and in the second configuration, the outer surface includes a micro pattern.

In some embodiments, a catheter and stent combination has a delivery configuration. The combination includes a catheter comprising an expandable balloon. The stent includes a stent body and a coating over at least a portion of the stent body. And, the coating includes a shape-memory polymer defining an outer surface. The outer surface of the shape memory polymer defines a first configuration and a second configuration. In the first configuration, the outer surface is smooth and in the second configuration the outer surface includes a micro pattern. Further, in the delivery configuration, the stent is disposed over at least a portion of the expandable balloon.

In some embodiments, the expandable balloon comprises a conductive covering and the stent is disposed around the conductive covering in the delivery configuration.

In some embodiments, such that the catheter applies heat to the stent, for example an inner lumen of the stent, to activate the shape-memory polymer, thereby encouraging the stent to resist, impede, or prevent stent migration.

In some embodiments, a catheter and stent combination comprises a catheter including a heating wire. The stent includes a stent body and a coating over at least a portion of the stent body. The coating comprises a shape-memory polymer defining an outer surface. The outer surface of the shape memory polymer defines a first configuration and a second configuration. In the first configuration the outer surface is smooth and in the second configuration the outer surface includes a micro pattern.

In some embodiments, the heating wire extends into the stent.

In some embodiments, the catheter and stent combination has a delivery configuration and an actuated configuration, wherein, in the delivery configuration, the heating wire is disposed within the stent in a helical or corkscrew shaped configuration and, upon application of heat via the heating wire, the catheter and stent combination transitions to the actuated configuration wherein the outer surface takes on the second configuration.

In some embodiments, a probe and/or basket made at least partially of conductive material, contacts and applies heat to an inner lumen of the stent. The stent, in turn, has a coating or covering comprising a bioadhesive thermoplastic or shape memory material. Upon application of heat to the bioadhesive thermoplastic or shape memory material, it changes shape. The bioadhesive thermoplastic or shape memory coating or covering can be activated along the stent's length at defined circumferential intervals or the entire length of the stent.

In some embodiments, a stent comprises a stent body and a mucoadhesive coating over at least a portion of the stent body. The mucoadhesive coating is formed from a polymeric material and defines a micro pattern. The micro pattern defines a plurality of holes extending through the mucoadhesive coating and the holes have a cross section less than or equal to 100 nanometers.

In some embodiments, the micro pattern is arranged in a repeating pattern along a surface of the mucoadhesive coating and at least 75% of the holes are spaced between 1 and 100 microns apart.

The above summary of some embodiments is not intended to describe each disclosed embodiment or every implementation of the present disclosure. The Figures, and Detailed Description, which follow, more particularly exemplify these embodiments.

U.S. Publication No. 2013/0268063, titled, “Anti-migration Micropatterned Stent Coating,” having inventors Laura Elizabeth Firstenberg, Claire M. McLeod, Shannon Taylor, Andrea Lai, and Sandra Lam, which was filed on Apr. 6, 2013, is herein incorporated by reference in its entirety.

U.S. Publication Nos. 2009/0098176 (“Medical devices with triggerable bioadhesive material”) and 2009/0317483 (“Bicomponent Bioadhesive for Biomedical Use”), and PCT Publication No. WO2012042522 (“Bioadhesive composition and device for repairing tissue damage”) are also incorporated by reference.

### BRIEF DESCRIPTION OF THE DRAWINGS

A detailed description of the invention is hereafter described with specific reference being made to the drawings.

FIG. 1 is a schematic view of an embodiment of a stent including a coating in a first configuration.

FIG. 2 is a schematic view of the embodiment of FIG. 1 with the coating in a second configuration.

FIG. 3A is a schematic view of a combination of an embodiment of a catheter and stent.

FIG. 3B is a cross-sectional view of the embodiment of FIG. 3A.

FIG. 4 is a sectional view of an embodiment of a catheter and stent.

FIG. 5 is a schematic view of an embodiment of a stent within a body lumen.

FIG. 6 is a top-down view of a micro pattern of FIG. 5.

FIGS. 6A-6C show a combination of an embodiment of a catheter and a stent in a delivery configuration, expanded configuration, and actuated configuration, respectively.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

References in the specification to “an embodiment”, “some embodiments”, “other embodiments”, etc., indicates that an embodiment includes a particular feature, structure, or characteristic, but every embodiment may not necessarily include the particular feature, structure, or characteristic. Moreover, such phrases do not necessarily refer to the same embodiment. Further, when a particular feature, structure, or characteristic is described in connection with an embodiment, it should be understood that such feature, structure, or characteristic may also be used in connection with other embodiments, whether or not explicitly described, unless clearly evidenced or stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Some embodiments of the present disclosure are directed to a stent, while other embodiments are directed to a combination of a catheter and a stent. In some embodiments, the stent is configured to transition in a controlled manner between a first configuration (e.g., smooth state) and a second configuration (e.g., micropatterned state).

In some embodiments, the catheter comprises an expandable balloon including a conductive covering. The conductive covering is configured to heat an inner surface of the stent. The stent, in turn, is disposed over the conductive covering.

In some embodiments, the catheter includes a probe or basket made from a conductive material. In some embodiments, the catheter includes a heating wire that expands into a helical or corkscrew shape.

In some embodiments, the stent includes a stent body and coating over at least a portion of the stent body. In some embodiments, the coating extends over all of the stent body. The coating can also extend over a portion or portions of the stent body. In some embodiments, the coating includes a shape-memory polymer defining an outer surface such that the outer surface is configured to transition from a first configuration to a second configuration. In the first configuration, the outer surface is smooth and, in the second configuration, the outer surface has a micro pattern. In some embodiments, the outer surface transitions from the first configuration to the second configuration upon application of heat to the shape-memory polymer coating.

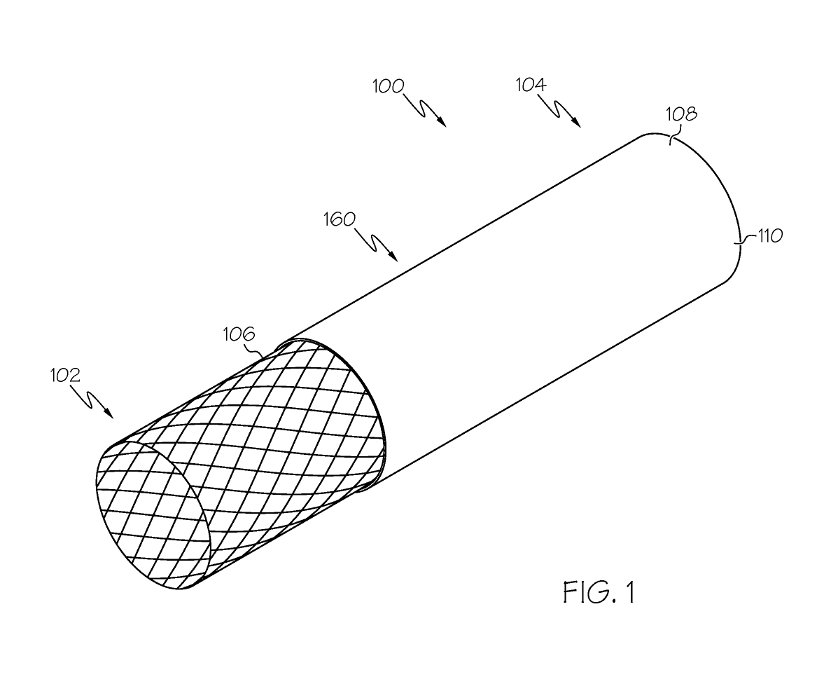

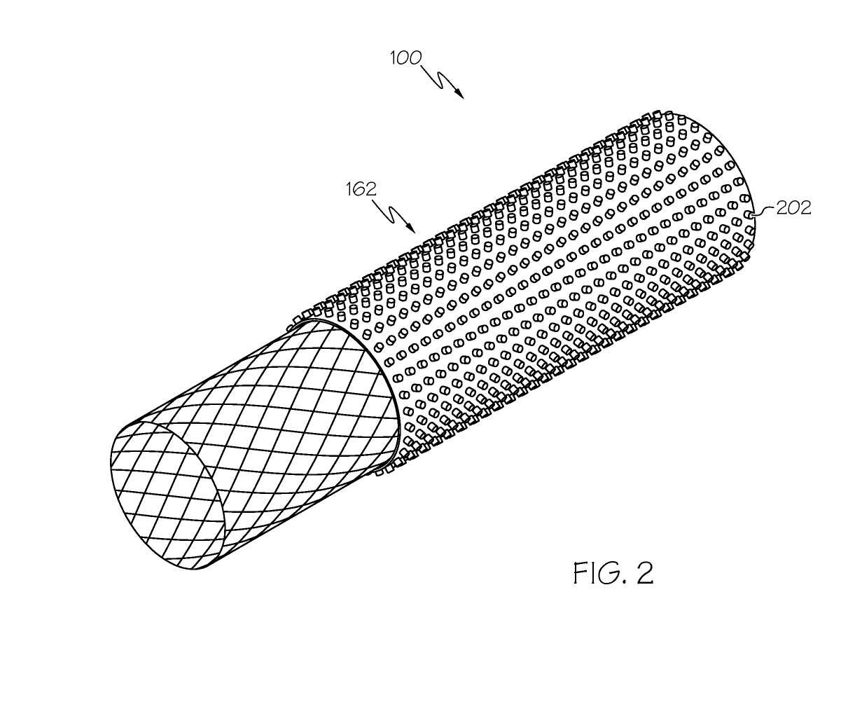

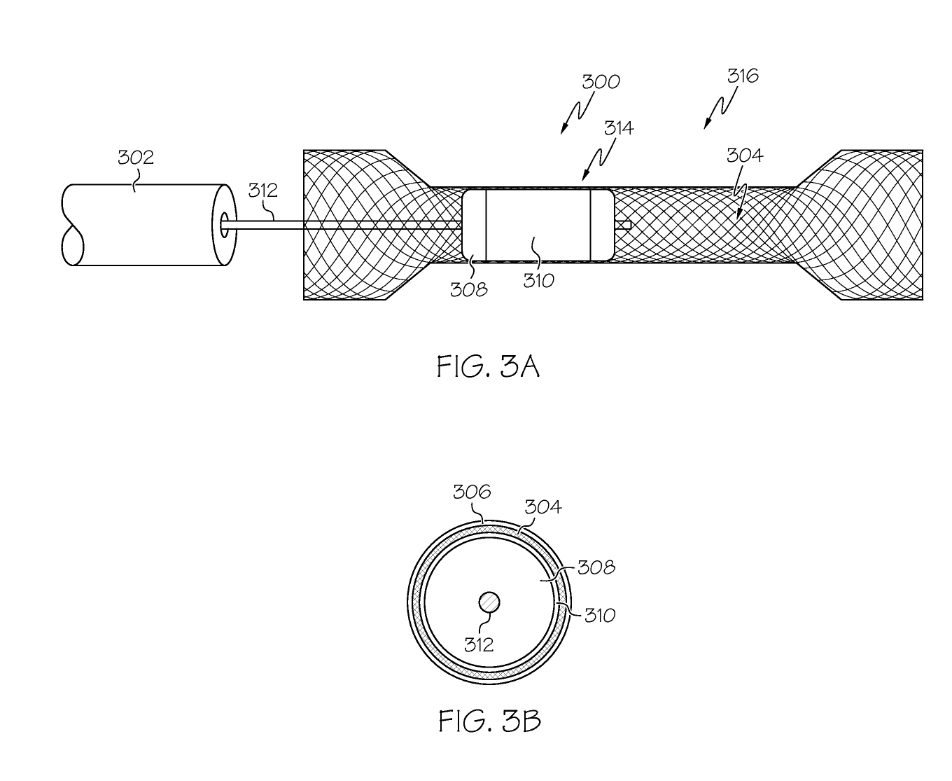

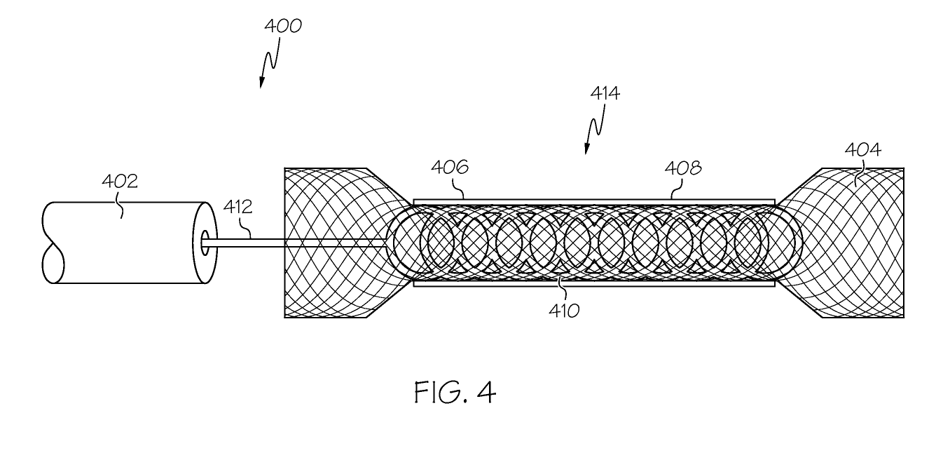

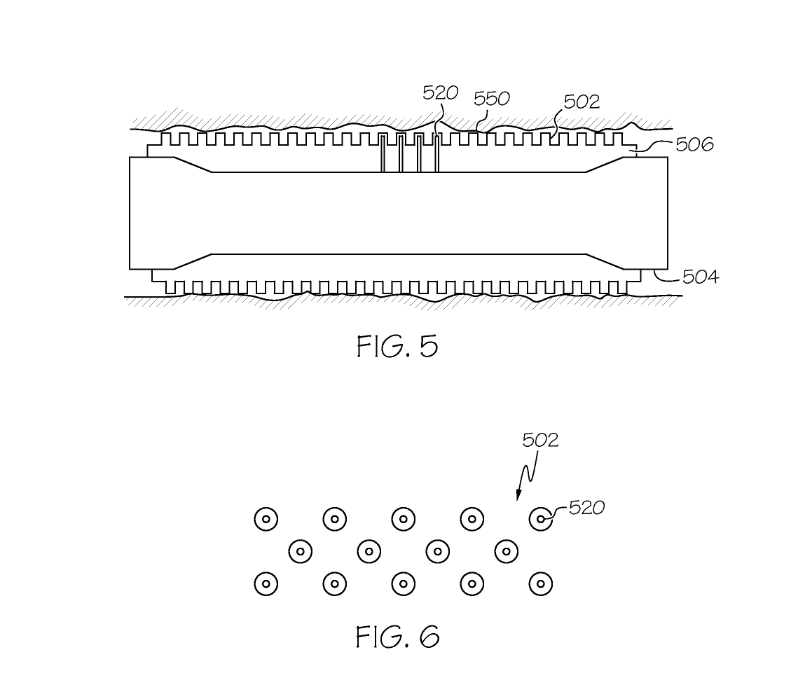

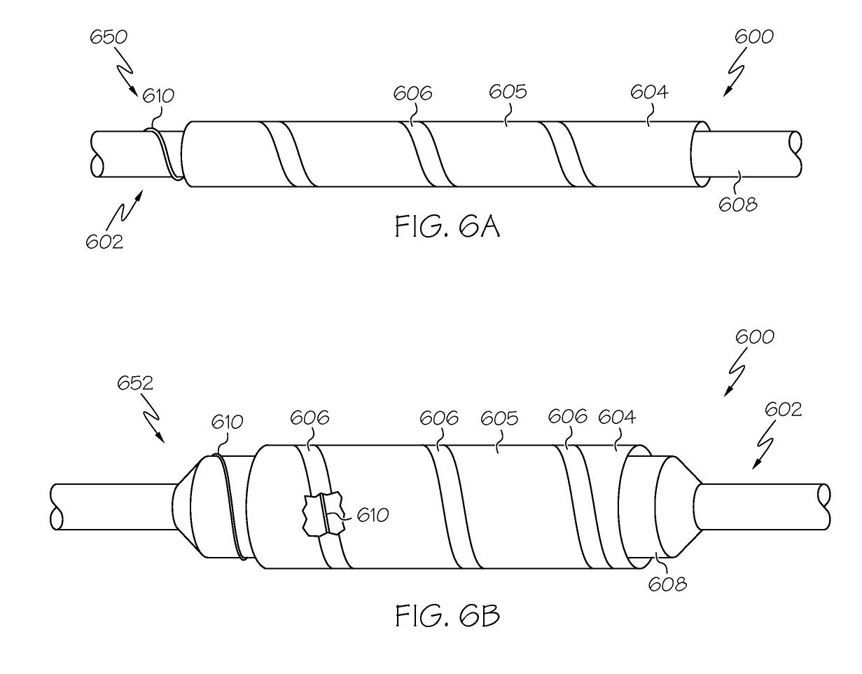

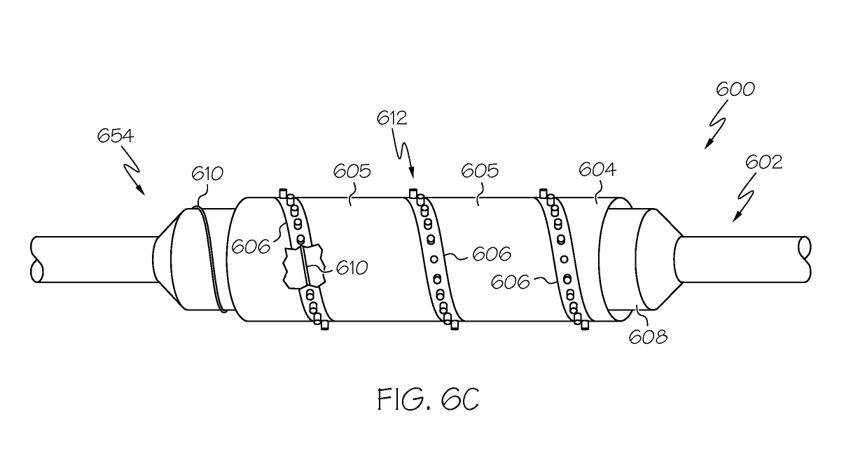

FIGS. 1 and 2 depict a stent 100 including an anti-migration feature, as discussed in greater detail below. FIGS. 3A and 3B illustrate a combination of a catheter 302 and a stent 304 having a temperature transfer system 314 including a balloon. FIG. 4 depicts a combination of a catheter 402 and a stent 404 having a temperature transfer system 414 including a conducting wire. FIG. 5 depicts a stent within a body lumen and FIG. 6 is a top-down view of a micro pattern of FIG. 5.

As shown in FIG. 1, the stent 100 includes a proximal end 102 and a distal end 104. The stent 100 also includes a stent body 106 extending longitudinally from the proximal end 102 to the distal end 104. The stent 100 includes an inner surface defining a lumen. Further, at least a portion of the stent body 106 has thereover a coating 108. The coating 108 and stent 100 are shown, in FIG. 1, in a partially offset manner, for the purposes of illustration. In some embodiments, the coating 108 is disposed over at least a portion of the stent body 106. The coating 108 can also be disposed along any desired length or portion of the stent body 106, for example, from the proximal end 102 to the distal end 104. In some embodiments, the coating 108 is formed entirely from a bio adhesive polymer or shape-memory polymer. In some embodiments, the coating 108 is partially formed from the bioadhesive polymer and/or shape-memory polymer.

Examples of suitable materials for the coating 108 include, but are not limited to, known and/or later developed shape memory polymers, thermoplastic bioadhesive polymers, and the like. According to the nature or properties of the shape memory polymer, for example, the stent coating 108 may display non adhesive properties until activated by one or more stimuli, such as heat. Once activated by heat, for example, the coating 108 may interact, interlock, and/or bond with tissue to adhere to adjacent tissue.

Examples of the suitable thermoplastic bioadhesive polymers having a triggerable bioadhesive property include, but are not limited to, acid polymers such as polymers containing methacrylic acid and/or acrylic acid, styrene-isobutylene-copolymers, polyurethane and its copolymers, silicone and its copolymers (e.g., polysiloxanes and substituted polysiloxanes), ethylene-alphaolefin copolymers, acrylic polymers and copolymers, polymethacrylates, polyacrylimides, vinyl halide polymers, polyvinylidene halides, polyvinyl ethers, polyvinylidene halides, polyvinyl ketones, polyvinyl aromatics, copolymers of vinyl monomers, copolymers of vinyl monomers and olefins such as ethylene-methyl methacrylate copolymers, polyamides, alkyd resins, polycarbonates, polyoxymethylenes, ethylene-vinyl acetate copolymers, polyamides, polyimides, polyethers, epoxy resins, alkyd resins, polyurethanes, thermoplastic elastomers, polyolefins, cellulosics, polyamides, polyesters, polysulfones, polytetrafluorethylenes, fluorosilicones, polycarbonates, acrylonitrile-styrene copolymers, ABS (acrylonitrile-butadiene-styrene) resins, acrylonitrile butadiene styrene copolymers, acrylics, polylactic acid, polylactic acid-polyethylene oxide copolymers, polycarbonates, polysaccharides, phospholipids, gelatins, cellulose ethers, collagens, chitosans, and chitins, or a combination of the foregoing.

Examples of suitable shape-memory polymers include covalently cross-linked semi-crystalline networks including, but not limited to, semi-crystalline rubbers, liquid-crystal elastomers and hydrogels containing phase separated crystalline microdomains.

In some embodiments, the coating 108 includes an outer surface 110 comprising a shape memory polymer. Further, in some embodiments, the outer surface 110 transitions from a first configuration 160 to a second configuration 162. In at least some embodiments, the outer surface 110 transitions from the first configuration to the second configuration upon application of heat to the coating 108. As shown for example in FIG. 1, the outer surface 110 is in the first configuration, where the outer surface 110 is smooth. In some embodiments, a smooth surface is desirable while inserting the stent 100 in unexpanded or collapsed state into a lumen within the patient's body so as to avoid any injury to body tissues. In at least some embodiments, the outer surface 110 remains smooth until heat is applied.

In some embodiments, the stent body 106 is formed of one or more strands arranged in a suitable pattern or interwoven or braided with each other. In some embodiments, the stent body 106 includes a monofilament or multi-filament structure. In some embodiments, the stent body 106 is balloon-expandable. The stent body 106 can also be self-expanding. The stent body 106 may be formed using suitable methods such as, but not limited to, weaving, braiding, welding, laser cutting, casting, extruding, and so forth. In some embodiments, the stent body 106 is monolithically or unitarily formed. The stent 100 can be delivered in an unexpanded state to a desired location within a lumen of the patient's body, and subsequently expanded by an internal radial force.

The stent 100 can have any desirable shape. For example, in some embodiments, the stent 100 has a non-uniform diameter along its length (e.g., the stent tapers, has one or more flared ends, etc.); in some embodiment, the stent 100 has a uniform diameter along its length. The stent 100 can have a circular or non-circular (e.g., ovoid) cross-section.

Further, in some embodiments, the stent body 106 is formed using suitable biocompatible metals and/or polymers. Examples of suitable materials for the stent 100 may include polyurethane (PU), polyethylene (PE), polytetrafluoroethylene (PTFE), or expanded polytetrafluoroethylene (ePTFE). In some embodiments, textile or fabric constructions including PTFE or ePTFE yarns, filament extrusions, or mesh may also be employed for the stent body 106. In addition to the polytetrafluoroethylene (PTFE/ePTFE) as mentioned above, examples of suitable biocompatible polymers also include, and are not limited to, polyolefins such as high density polyethylene (HDPE) and polypropylene (PP), polyolefin copolymers and terpolymers, polyethylene terephthalate (PET), polyesters, polyamides, polyurethaneureas and polycarbonates, polyvinyl acetate, thermoplastic elastomers including polyether-polyester block copolymers, polyvinyl chloride, polystyrene, polyacrylate, polymethacrylate, polyacrylonitrile, polyacrylamide, silicone resins, combinations and copolymers thereof, and the like. Further, in some embodiments, the stent body 106 includes materials made from or derived from natural sources, such as, but not limited to collagen, elastin, glycosaminoglycan, fibronectin and laminin, keratin, alginate, and combinations of these.

In some embodiments, the stent body 106 comprises a suitable material having enhanced external imaging properties under magnetic resonance imaging (MRI) and/or ultrasonic visualization techniques. Examples of the materials for enhancing MRI visibility include, but are not be limited to, metal particles of gadolinium, iron, cobalt, nickel, dysprosium, dysprosium oxide, platinum, palladium, cobalt based alloys, iron based alloys, stainless steels, or other paramagnetic or ferromagnetic metals, gadolinium salts, gadolinium complexes, gadopentetate dimeglumine, compounds of copper, nickel, manganese, chromium, dysprosium and gadolinium. Similarly, to enhance the visibility under ultrasonic visualization, the various components of the stent 100 may include ultrasound resonant material, such as, but not limited to, gold. Further, in some embodiments, the stent body 106 includes radiopaque materials, such as metallic-based powders or ceramic-based powders, particulates or pastes which may be incorporated into the polymeric material of the stent body 106. Other metallic complexes that are useful as radiopaque materials are also contemplated. In at least some embodiments, the radiopaque material is disposed over the stent body 106 at selectively desired areas along the stent 100. Further, in some embodiments, the radiopaque material is disposed over the entirety of the stent body 106; the radiopaque material can be arranged in any suitable way, depending on the desired end-product and application.

In some embodiments, portions of the stent body 106, for example strands of the stent 100, respectively, have an inner core of iridium and an outer member or layer of nitinol; such composite strands or strand portions provide enhanced radiopacity or visibility. In some embodiments, a radiopaque material is blended with the polymer composition from which a polymeric strand of the stent 100 is formed, and subsequently fashioned into the stent body 106 as described herein. The radiopaque material can also be applied to the surface of the metal or polymer strands. Various radiopaque materials and their salts and derivatives may be used including, without limitation, bismuth, barium and its salts such as barium sulfate, tantalum, tungsten, gold, platinum and titanium, and so forth. Further, the stent body 106 can be formed from any desirable material, for example polymeric or metallic. In some embodiments, the stent body 106 is formed from nickel, titanium, nickel-titanium alloy, stainless steel, cobalt, platinum, and suitable combinations and alloys thereof. The skilled artisan will appreciate that other metals can also be used.

FIG. 2 shows the outer surface 110 of the coating 108 in the second configuration, e.g., having a micro pattern 202. As mentioned above, in some embodiments, the outer surface 110 develops the micro pattern 202 upon heating the coating 108. In some embodiments, heat is applied to the inner surface of the stent 100 to heat the coating 108. The micro pattern 202 formed on the outer surface 110 of the coating 108 improves adhesion of the stent 100 to the tissue within the patient's body. Hence, the coating 108 reduces, impedes, or prevents migration of the stent 100 from the desired location within the patient's body. In some embodiments, the coating 108 is formed from a bioadhesive thermoplastic material which forms the micro pattern 202 and sticks to the tissue when heat is applied to the coating 108. In some embodiments, the outer surface 110 is also configured to transition from the second configuration back to the first configuration (e.g., smooth configuration) when the stent is cooled. Further, in some embodiments, the bioadhesive thermoplastic material is configured to display non-adhesive properties until activated by the heat.

In some embodiments, the coating 108 is made solely from a bioadhesive thermoplastic material. In some embodiments, however, the coating 108 includes the bioadhesive thermoplastic material and one or more additional materials. After the stent 100 is deployed, in some embodiments, an endoscopic tool may be used to generate and apply heat to the inner wall of the stent 100 such that the heat is translated to the coating 108. Consequently, the thermoplastic material of the coating 108 adheres to the lumen wall, anchoring the stent 100 in place and preventing the stent 100 from migrating.

Any suitable method of activating the material of the coating 108 to transition between the first and second configurations can be employed. In some embodiments, the coating 108 is heat activated. In some embodiments, the coating 108 is activated via UV or other light. In some embodiments, an electrical current is used to activate the coating 108. In some embodiments, the heat is provided by the patient's natural body heat. In some embodiments, heat, light, and/or electrical current is applied using an external source, such as the one explained with reference to FIGS. 3A, 3B, and 4.

In some embodiments, the stent 100 comprises a therapeutic agent that is released into the body over time. In some embodiments, the stent body 106 comprises the therapeutic agent and, in some embodiments, the coating 108 comprises the therapeutic agent. In some embodiments, both the stent body 106 and coating 108 comprise one or more therapeutic agents, which can be the same therapeutic agent or different agents. Examples of the useful therapeutic agents include, but are not limited to, anti-platelets, anti-thrombins, anti-tumor drugs, anti-hyperplasia agents, anti-plaque building agents, cytostatic agents, and antiproliferative agents, or other drugs for a specific purpose. This may also include agents for gene therapy. The foregoing list of therapeutic agents is provided by way of example and is not intended to be limiting, as other therapeutic agents and drugs may be developed which are equally applicable for use with the present invention.

FIG. 3A is a schematic view of a combination 300 of a catheter 302 and a stent 304. As shown, the catheter 302 can comprise an endoscope. As further shown, the catheter 302 includes a temperature transfer system 314. The temperature transfer system 314 includes an expandable balloon 308 and a shaft 312. In some embodiments, the balloon 308 includes a conductive covering 310 of suitable electrical and/or heat conductor such as metal. In some embodiments, the conductive covering 310 is located over the portions of the balloon 308 that contact the interior of the stent 304. In some embodiments, the balloon 308 includes conductive covering 310 around a portion of the circumference of the balloon 308 or around or along specific sections of the balloon 308. For example, the conductive covering 310 may be disposed or arranged in a number of adjacent circumferential rings over the balloon 308. In some embodiments, the conductive covering 310 is heated or cooled using an external source. The conductive covering 310 is configured to be in contact with the inner lumen of the stent 304 and apply or transfer the heat to an inner surface of the stent 304. The stent 304 can be inserted into the lumen simultaneously with the balloon 308, for example where the stent 304 is positioned around the balloon 308 in an unexpanded configuration. In some embodiments, the stent 304 is inserted into the lumen prior to the balloon 308. After insertion of the stent 304, the balloon 308 is moved into position within the stent 304, for example a self-expanding stent, and the conductive covering 310 is heated, thereby causing the stent 304 to transition from the first configuration to the second configuration, as previously described.

The stent 304 includes a coating 306 of shape-memory polymer or a bioadhesive thermoplastic material over at least a portion of stent body. In some embodiments, for example where the coating 306 is a shape-memory polymer, the outer surface of the coating 306 is configured to transition between a first configuration and a second configuration. In some embodiments, the outer surface is smooth in the first configuration. The outer surface transitions to a second configuration when heated by the balloon 308. In some embodiments, the balloon 308 is configured to aid in expansion of the stent 304 and also to apply heat to the inner surface of the stent 304 for activating the stent's coating 306.

In some embodiments, the balloon 308 is disposed inside the stent 304 during delivery of the stent 304 to the treatment site. In some embodiments, however, the stent 304 is first delivered and the balloon 308 is moved into position within the stent 304 to apply heat or other stimuli (e.g., UV light, electrical current). Properly situated within the stent 304, the balloon 308 is radially inflated. Then, in order to transition the coating 306, the conductive covering 310 of the balloon 308, for example, is heated. Where the coating 306 transitions from the first configuration to the second confirmation via electric current or UV light, for example, the balloon or other device can be operated accordingly.

In some embodiments, the coating 306 comprises a shape memory polymer which, when in the second configuration, has a micro pattern defined by a number of micro pillars. The micro pillars may be in form of cylinders, rectangular prisms, or any other suitable structure. In some embodiments, the micro pattern comprises a plurality of protrusions, which extend over one or more portions of the outer surface of the coating 306 when the coating 306 is in the second configuration, for example upon heating the inner surface of the stent body.

In some embodiments, the coating 306 has holes which are packed with a secondary material different from a primary material of the coating. Upon application of heat, UV, or other stimuli (as discussed herein), the secondary material degrades and exits the holes, leaving the primary material behind. In this way, the remaining primary material adheres to the adjacent tissue.

FIG. 3B is a cross-sectional view 316 of the balloon 308 of FIG. 3A. As further shown in FIG. 3B, the catheter shaft 312 passes longitudinally through the balloon 308. In some embodiments, a conductive covering 310 extends circumferentially around the entirety of the balloon 308. The conductive covering 310 can also extend along or around only portions of the balloon 308 in any desirable configuration.

FIG. 4 is a side view of an embodiment of a combination 400 of a catheter 402 and a stent 404. In some embodiments, the stent 404 is similar to the stent 100 in structure and function as discussed with reference to FIGS. 1 and 2. The stent 404 comprises a coating 406 defining an outer surface 408. In some embodiments, the coating 406 comprises a thermoplastic bioadhesive polymer and/or shape-memory polymer. In some embodiments, the thermoplastic polymer is thermo sensitive to dielectric heating. In some embodiments, the thermoplastic polymer is sensitive to high frequency alternating current. In some embodiments, the dielectric heating may be caused by dipole rotation. The thermoplastic polymer may be a synthetic or semi synthetic. Further, the outer surface 408 of the coating 406 may define a first configuration and a second configuration. In the first configuration, the outer surface 408 of the coating 406 is smooth. In some embodiments, in the second configuration, the outer surface 408 of the coating 406 forms a micro pattern.

In some embodiments, the catheter 402 includes a heat transfer system 414. The heat transfer system 414 includes a shaft 412 and a heating wire 410 having a suitable structure, for example, but not limiting to, a helical or corkscrew configuration. In some embodiments, the heating wire 410 expands into a corkscrew shape and is configured to apply heat to the inner surface of the stent 404, thereby activating the coating 406 of the stent 404. In some embodiments, portions of the stent 404 are in contact with the heating wire 410 and only those portions are activated, for example, to take on a micro pattern (e.g., in the case of a shape memory polymer) or adhere to the lumen (e.g., in the case of a thermoplastic bioadhesive material). In some embodiments, however, localized heating or stimulation of portions of the stent can cause the entirety of the stent to transition from the first configuration to the second configuration, depending upon the material properties of the stent and/or coating 406.

In some embodiments, the catheter 402 forms a unitary structure with the temperature transfer system 414; the catheter 402 and the temperature transfer system 414 can also be two or more separate structures, and may be joined, for example, via the shaft 412.

In some embodiments, the heat transfer system 414 is disposed or deployed within the stent 404 from a distal end of the stent 404. As shown, the heating wire 410 is disposed within the stent 404 in a helical configuration. In some embodiments, the heat may be at or above the body temperature, depending on the polymer formulation of the coating 406. The micro pattern may facilitate interlocking of the outer surface of the stent 404 with the tissue, thereby facilitating an anti-migration mechanism.

Further, in some embodiments, for example when required or otherwise advantageous, the stent 404 can be rendered removable by cooling the stent body of the stent 404 below the body temperature to transition the stent from the second configuration back to the first configuration (e.g., from the micro pattern state to smooth state). In this way, the stent 404 may be cooled using the temperature transfer system, for example as illustrated via reference numerals 300 or 400.

In some embodiments, the balloon and heating wire are replaced with a probe or basket made at least partially of a conductive material, the probe is configured to contact and heat the inner lumen of the stent. The basket of some of these embodiments is similar in design to a related art bronchial thermoplasty device. In some embodiments, the coating (e.g. 106, 306, 406) is be activated along the stent's length at defined circumferential intervals.

In some embodiments, for example as shown in FIG. 5, the stent 504 has a coating 506, for example a mucoadhesive coating, defining a micro pattern 502 extending from the surface of the coating 506. In some embodiments, the coating 506 extends over a portion of the stent body; the coating 506 can also extend over the entirety of the stent body.

In some embodiments, the coating 506 is formed from a polymeric material. The micro pattern 502 can be of any desirable configuration. In some embodiments, for example, the micro pattern 502 is arranged in a repeating pattern, as shown in FIG. 6, which illustrates a top-down view of a portion of the coating 506.

In some embodiments, the micro pattern 506 defines a number of holes 520 extending through the coating 506. The holes 520 have a cross-section less than or equal to 100 nanometers. In some embodiments, the holes 520 have a circular cross-section having a diameter less than or equal to 100 nanometers. In some embodiments, at least 75% of the holes 520 are spaced between 1 and 100 microns apart. In some embodiments, at least 90% of the holes 520 are spaced between 1 and 100 microns from any adjacent hole 520.

In some embodiments, the coating 506 includes thiolated chitosan molecules, polymers of ethyl acetate such as Carbopol 971P NF, and/or polycarbophil. In some embodiments, the holes 520 are unevenly distributed on the coating 506 or stent body; in some embodiments, however, the holes 520 are evenly distributed on the coating 506 or stent body.

The coating 506, for example a mucoadhesive coating, may provide adhesion to the lumen wall 550, thereby reducing the risk of stent migration. Further, tissue ingrowth into the holes 520 anchors the stent 504 as nanofiberous tubes grow into the holes 520 of the stent 504. Where the holes 520 have a cross-section less than approximately 100 nanometers, the cross-section of the nanofiberous tubes is limited or “bottlenecked” by the size of the holes 520. The limited size of the ingrown nanofiberous tubes, in turn, permits the stent 504 to be removed, if necessary, without triggering a sensory output to the patient. Consequently, removal of the stent 504 would not be traumatic.

In some embodiments, for example where a mucoadhesive coating is used, the stent need not make use of a shape-memory polymer. Instead, in such embodiments, the stent can be inserted into a lumen with the micro pattern already deployed. Of course, the holes 520 can also be used in combination with any suitable shape memory polymer or configuration.

With regard to FIGS. 6A-6C, an embodiment of a combination 600 of a catheter 602 and a stent 604 is shown in various configurations. In FIG. 6A, the combination 600 is illustrated in a delivery configuration 650, wherein the stent 604 is unexpanded and the expandable balloon 608 is also unexpanded. In FIG. 6B, the combination 600 is shown in an expanded configuration 652, with the expandable balloon 608 and stent 604 being expanded. Finally, in FIG. 6C, the combination 600 is shown in an actuated configuration 654, wherein the expandable balloon 608 and stent 604 have been expanded and the micro pattern 612 is deployed.

As illustrated in FIGS. 6B and 6C, the stent 604 is shown with a cutaway to illustrate the conductive covering 610. As shown, the conductive covering 610 is disposed over portions of the expandable balloon 608, for example in strips or bands around the expandable balloon 608. As will be appreciated, the conductive covering 610 can have any desirable configuration; it can cover the entire expandable balloon 608 or only portions thereof.

In some embodiments, the stent 604 has a coating 606 over at least a portion of the stent body 605. In some embodiments, the coating 606 is formed from one or more shape memory polymers, as discussed above. Moreover, in some embodiments, the location of the coating 606 corresponds with the location of the conductive covering 610 such that strips of conductive covering 610 heat (or cool) the shape memory polymer of the coating 606, thereby causing it to form the micro pattern 612.

A description of some embodiments of the heat treatments is contained in one or more of the following numbered statements:
Statement 1. A stent comprising:

a stent body; and

a coating over at least a portion of the stent body, the coating comprising a shape-memory polymer defining an outer surface, the outer surface having a first configuration and a second configuration;

in the first configuration the outer surface being smooth and in the second configuration, the outer surface having a micro pattern.
Statement 2. The stent of statement 1, wherein the stent body is formed from a polymeric material.
Statement 3. The stent of any one of the preceding statements, wherein the stent body is formed from a metallic material.
Statement 4. The stent of any one of the preceding statements, wherein the stent body is self-expanding.
Statement 5. The stent of any one of the preceding statements, wherein the stent body is balloon-expandable.
Statement 6. The stent of any one of the preceding statements, wherein the outer surface of the shape-memory polymer transitions from the first configuration to the second configuration upon application of heat to the shape-memory polymer.
Statement 7. The stent of any one of the preceding statements wherein the micro pattern defines a plurality of holes extending through the coating, the holes having a cross-section of about 100 nanometers or less.
Statement 8. The stent of any one of the preceding statements the micro pattern is a repeating pattern along the surface of the coating and at least 75% of the holes are spaced between about 1 and 100 microns apart.
Statement 9. A catheter and stent combination having a delivery configuration and comprising:

a catheter and a stent, the catheter having an expandable balloon;

the stent having a stent body and a coating over at least a portion of the stent body, the coating comprising a shape-memory polymer defining an outer surface, the outer surface having a first configuration and a second configuration; and

in the first configuration the outer surface being smooth and in the second configuration, the outer surface having a micro pattern;

wherein, in the delivery configuration, the stent is disposed over at least a portion of the expandable balloon.
Statement 10. The catheter and stent combination of statement 9, wherein the expandable balloon comprises a conductive covering and the stent is disposed around the conductive covering in the delivery configuration.
Statement 11. The catheter and stent combination of statement 10, wherein the outer surface of the shape-memory polymer transitions from the first configuration to the second configuration upon application of heat to the shape-memory polymer via the conductive covering.
Statement 12. The catheter and stent combination of statement 10, wherein the conductive covering is arranged in a plurality of adjacent circumferential rings.
Statement 13. The catheter and stent combination of statement 10 or 11, wherein, in the second configuration, the micro pattern is defined by a plurality of micro pillars.
Statement 12. A catheter and stent combination comprising:

a catheter and a stent, the catheter having a heating wire;

the stent having a stent body and a coating over at least a portion of the stent body, the coating comprising a shape-memory polymer defining an outer surface, the outer surface having a first configuration and a second configuration;

in the first configuration the outer surface being smooth and in the second configuration, the outer surface having a micro pattern.
Statement 13. The catheter and stent combination of statement 12 having a delivery configuration and an actuated configuration, wherein, in the delivery configuration, the heating wire is disposed within the stent in a helical configuration; and

upon application of heat via the heating wire, the catheter and stent combination transitions to the actuated configuration wherein the outer surface takes on the second configuration.
Statement 14. A stent comprising:

a stent body; and

a mucoadhesive coating over at least a portion of the stent body, the mucoadhesive coating formed from a polymeric material and defining a micro pattern, the micro pattern defining a plurality of holes extending through the mucoadhesive coating, the holes having a cross section less than or equal to 100 nanometers.
Statement 15. The stent of statement 14, wherein the micro pattern is arranged in a repeating pattern along a surface of the mucoadhesive coating and at least 75% of the holes are spaced between 1 and 100 microns from any adjacent hole
Statement 16. The any of the preceding statements wherein at least 90% of the holes are spaced between 1 and 100 microns from any adjacent hole.
Statement 17. The stent of claim 14, wherein the mucoadhesive coating comprises thiolated chitosan molecules, polymers of ethyl acetate, polycarbophil, and mixtures thereof.
Statement 18. The stent of any of the preceding statements, wherein the holes are evenly distributed along the stent body.
Statement 19. The stent of any of the preceding statements wherein the holes are packed with a secondary material.
Statement 20. The stent of statement 19 wherein the secondary material is a degradable material.

The embodiments or aspects of the stent and catheter as disclosed above, including the embodiment(s) presented in the claims, may be combined in any suitable fashion or combination.

It should be understood that this disclosure is illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A stent comprising:
a stent body; and
a coating over at least a portion of the stent body, the coating comprising a shape-memory polymer defining an outer surface, the outer surface having a first configuration and a second configuration;
in the first configuration the outer surface being smooth and in the second configuration, the outer surface having a micro pattern.

2. The stent of claim 1, wherein the stent body is formed from a polymeric material.

3. The stent of claim 1, wherein the stent body is formed from a metallic material.

4. The stent of claim 1, wherein the stent body is self-expanding.

5. The stent of claim 1, wherein the stent body is balloon-expandable.

6. The stent of claim 1, wherein the outer surface of the shape-memory polymer transitions from the first configuration to the second configuration upon application of heat to the shape-memory polymer.

7. The stent of claim 1 wherein the micro pattern defines a plurality of holes extending through the coating, the holes having a cross-section of about 100 nanometers or less.

8. The stent of claim 1 wherein the micro pattern is a repeating pattern along the surface of the coating and at least 75% of the holes are spaced between about 1 and 100 microns apart.

9. A stent delivery device comprising:
a catheter and a stent, the catheter having an expandable balloon;
the stent having a stent body and a coating over at least a portion of the stent body, the coating comprising a shape-memory polymer defining an outer surface, the outer surface having a first configuration and a second configuration; and
in the first configuration the outer surface being smooth and in the second configuration, the outer surface having a micro pattern;
wherein, in the delivery configuration, the stent is disposed over at least a portion of the expandable balloon.

10. The stent delivery device of claim 9, wherein the expandable balloon comprises a conductive covering and the stent is disposed around the conductive covering in the delivery configuration.

11. The stent delivery device of claim 10, wherein the outer surface of the shape-memory polymer transitions from the first configuration to the second configuration upon application of heat to the shape-memory polymer via the conductive covering.

12. The stent delivery device of claim 11, wherein the conductive covering is arranged in a plurality of adjacent circumferential rings.

13. The stent delivery device of 11, wherein, in the second configuration, the micro pattern is defined by a plurality of micro pillars.

14. A stent comprising:
a stent body; and
a mucoadhesive coating over at least a portion of the stent body, the mucoadhesive coating formed from a polymeric material and defining a micro pattern, the micro pattern defining a plurality of holes extending through the mucoadhesive coating, the holes having a cross section less than or equal to 100 nanometers.

15. The stent of claim 14, wherein the micro pattern is arranged in a repeating pattern along a surface of the mucoadhesive coating and at least 75% of the holes are spaced between 1 and 100 microns from any adjacent hole.

16. The stent of claim 15 wherein at least 90% of the holes are spaced between 1 and 100 microns from any adjacent hole.

17. The stent of claim 14, wherein the mucoadhesive coating comprises thiolated chitosan molecules, polymers of ethyl acetate, polycarbophil, and mixtures thereof.

18. The stent of claim 14, wherein the holes are evenly distributed along the stent body.

19. The stent of claim 14 wherein the holes are packed with a secondary material.

20. The stent of claim 19 wherein the secondary material is a degradable material.
